(19) United States
(12) Patent Application Publication
(10) Pub. No.: US 2009/0326397 A1
(43) Pub. Date: Dec. 31, 2009

(54) CLINICAL APPLICATIONS FOR ELECTRICAL TOMOGRAPHY DERIVED METRICS

(76) Inventors: Yashar Behzadi, San Francisco, CA (US); Timothy Robertson, Belmont, CA (US); Gregory Moon, Orinda, CA (US); Olivier Colliou, Los Gatos, CA (US)

Correspondence Address: Proteus Biomedical, Inc.;Bozicevic, Field & Francis LLP, 1900 University Avenue, Suite 200, East Palo Alto, CA, 94303, US

(21) Appl. No.: 12/492,672

(22) Filed: Jun. 26, 2009

Related U.S. Application Data
(60) Provisional application No. 61/076,582, filed on Jun. 27, 2008.
(60) Provisional application No. 61/164,679, filed on Mar. 30, 2009.

(51) Int. Cl. A61B 5/04

(52) U.S. Cl. 600/508

(57) ABSTRACT

Clinical applications for electrical tomography derived metrics are disclosed. One aspect of the present invention pertains to a method for generating clinical data by processing one or more metrics obtained via an electrical tomography. The method comprises receiving one or more metrics of an electrode stably associated with a tissue site of a subject, where the metrics are based on an induced signal of the electrode generated in response to one or more continuous electrical fields applied to the subject during an electrical tomography process. In addition, the method comprises generating clinical data of the internal organ of the subject based on the metrics.

### CROSS-REFERENCE TO RELATED APPLICATIONS

Pursuant to 35 U.S.C. §119(e), this application claims priority to U.S. Provisional Patent Application Ser. No. 61/076,582 filed on Jun. 27, 2008 and U.S. Provisional Patent Application No. 61/164,679 filed on Mar. 30, 2009, the disclosures of which are herein incorporated by reference.

### INTRODUCTION

In the field of medicine, evaluation of a property or characteristic associated with a tissue may be desirable for diagnostic or therapeutic purposes. Cardiac resynchronization therapy (CRT) may be an example where evaluation of cardiac tissue motion as observed by ultrasound techniques is often employed for diagnostic and therapeutic purposes.

For CRT, some tissue properties may be approximated via external measurements. In one example, external ultrasound measurements may be used to calculate various tissue parameters such as the hemodynamic parameter of change in pressure over time, dP/dt. The external ultrasound measurements may be used to observe cardiac wall motion directly. Tissue Doppler imaging (TDI), which uses ultrasound technology to examine the heart by determining the velocity and direction of tissue and/or blood flow utilizing the Doppler effect, may be the most frequently used technique to evaluate the time course of displacement of the septum, mitral valve annulus, and/or left ventricle free wall.

However, TDI has been limited to wall position determination via an external ultrasonography where a valve function, cardiac output or synchronization index may be measured. In addition, the patient who is undergoing the ultrasonic procedure may be typically observed in a supine position. Thus, the cardiac activity of the patient measured by the procedure may reflect this one position only. Accordingly, the ultrasound procedure may not be a viable tool for measuring cardiac parameters during dynamics activities, such as running, walking, etc.

Moreover, there are currently no useful clinically available means to accurately determine cardiac-related parameters on a substantially automatic, real-time, machine readable, and/or continuous basis. Further, and as a result of the lack of automatic, real time cardiac-related parameters, there are no accurate and continuous means to derive diagnostic, inferential, and/or predictive clinical data.

### BRIEF DESCRIPTION OF THE DRAWINGS

Aspects are illustrated by way of example and not limitation in the figures of the accompanying drawings, in which like references indicate similar elements and in which:

FIG. 1 illustrates a cross-sectional view of a heart with a cardiac timing device.

FIG. 2A illustrates an exemplary electrical tomography (ET) system.

FIG. 2B illustrates an exemplary process of calibrating a voltage to displacement conversion factor.

FIG. 2C illustrates an exemplary method for measuring a displacement of an electrode due to a change in induced voltage of the electrode.

FIG. 3 illustrates an exemplary data analysis process.

FIGS. 4a, 4b, and 4c illustrate two dimensional and three dimensional representations of clinical data.

FIG. 4d illustrates physiologically meaningful morphology of the principal velocity graph of FIG. 4c.

FIGS. 5a, 5b, 5c, and 5d illustrate an interpretation of an ET velocity trace of the principal velocity graph in FIG. 4d.

FIG. 5e illustrates a difference in corresponding time-to-peaks (TTPs) of two systolic velocity waves for two electrodes located on two different tissue sites in the heart.

FIG. 6 illustrates a relative position of electrodes derived from ET.

FIG. 7 illustrates a direction defined normal to the mitral annular plane toward the apex of a heart.

FIG. 8 illustrates interpretations of an exemplary ET velocity trace.

FIG. 9 illustrates an analysis of the impact of a left ventricle (LV) pacing location.

FIG. 10 illustrates a data comparison to dP/dT (max).

FIG. 11 illustrates an ET S-Velocity amplitude versus a pacing configuration.

FIG. 12 illustrates a correlation between ET S-Velocity amplitude and dP/dT (max) data.

FIG. 13a illustrates ET displacement data as a surrogate measure for an LV volume metric.

FIG. 13b illustrates other exemplary utilities of ET displacement data.

FIG. 14 illustrates ET S-Velocity data as a surrogate measure for a dP/dT (max) metric in an animal model.

FIG. 15 is a process flow chart of an exemplary method for generating clinical data based on measurements or metrics obtained during an electrical tomography.

Other features of the present aspects will be apparent from the accompanying drawings and from the detailed description that follows.

### DETAILED DESCRIPTION

Reference will now be made in detail to the aspects of the invention, examples of which are illustrated in the accompanying drawings. While the invention will be described in conjunction with the aspects, it will be understood that they are not intended to limit the invention to these aspects. On the contrary, the disclosure is intended to cover alternatives, modifications and equivalents, which may be included within the spirit and scope of the invention. Furthermore, in the detailed description, numerous specific details are set forth in order to provide a thorough understanding of the present disclosure; however, it will be obvious to one of ordinary skill in the art that the present disclosure may be practiced without these specific details. In other instances, well known methods, procedures, components, and circuits have not been described in detail as not to unnecessarily obscure aspects of the present invention.

Systems and methods for deriving physiologic parameters as well as clinical data for clinical applications are provided. The physiologic parameters, e.g., cardiac-related parameters, intestinal-related parameters, urinary system-related parameters, etc., may be generated by various methods including, for example, continuous field tomography. The clinical data may be derived from the cardiac-related parameters according to various methods and systems, examples of which are discussed hereafter in detail. The subject systems and methods find use in a variety of different clinical applications, such as cardiac-related applications, e.g., diagnostic and inferential applications predicated on performance and other physiologic metrics. Examples include measurements of left ventricle stiffness (LV stiffness) and heart size, diastolic dysfunction, proxies for other metrics, and clinical application thereof.

The term “metrics,” as used herein, refers to any measurement, characteristic, property, calculation, or the like associated with human or non-human tissue, e.g., an evaluation of tissue location motion, such as of a cardiac location of a heart wall. The term “tissue”, as used herein, refers to any ensemble of animal tissue, e.g., a specific tissue site, an organ, etc.

In continuous field tomography, a continuous field, e.g., an electrical field, sensing element is stably associated with a tissue location, and a property of, e.g., a change in, the continuous field sensed by the sensing element is employed for evaluation purposes, e.g., identification and measurement of tissue movement. Various methods and devices associated with continuous field tomography, as well as data derived thereby, are described in PCT Patent Application Serial No. PCT/US2005/036035 (WIPO publication no. wo/2006/042039) filed on Oct. 6, 2005, also filed as U.S. patent application Ser. No. 11/664,340 (published U.S. patent application no. 20080183072) filed on Mar. 30, 2007, U.S. patent application Ser. No. 11/731,786 (published U.S. patent application no. 20080058656) filed on Mar. 30, 2007, and U.S. patent application Ser. No. 11/731,726 filed on Mar. 30, 2007, each of the foregoing herein incorporated by reference in its entirety.

Aspects of the present invention can derive metrics using several types of continuous fields. For example, a tomography system may apply an electrical field, a magnetic field, or a pressure field, e.g., using acoustic waves, as a continuous field. In general, a dynamic field operating at a given frequency can be a traveling wave or a standing wave. The field is typically a vector quantity, whereas the field magnitude is often a scalar. Without losing generality, the field magnitude can be expressed as:

F₀=A·sin(2π·f·t+φ)

where A is the field amplitude, f is the frequency at which the field oscillates, t is the time, and φ is the phase shift.

When a tissue region is subjected to such a field and when a sensing element, such as an electrode, resides in the same region, e.g., by being stably associated therewith, the field can induce a signal upon the sensing element. The induced signal may be of the form:

S=B·sin(2π·f′·t+φ)

where B is the amplitude of the induced signal, f′ is the induced signal's frequency, and φ′ is the induced signal's phase shift. In certain aspects of interest is a transformation function “T”, which can be determined from S and Fₒ using the following relationship: S=T(x,y,z,t)^{o}Fₒ. In these aspects, tissue location movement may be evaluated by detecting a transformation of the continuous field. Because B, f′, and φ′ may depend upon the sensing element's location or movement in the field, one can perform tomography based on one or more of these values.

For example, if a continuous electrical field driven by an alternating-current (AC) voltage is present in a tissue region, an induced voltage may be detected on an electrode therein. The frequency of the induced voltage, f′, is the same as the frequency of the electrical field. The amplitude of the induced signal, however, varies with the location of the electrode. By detecting the induced voltage and by measuring the amplitude of the signal, the location as well as the velocity of the electrode can be determined.

A magnetic field can achieve a similar result. For example, an AC sinusoidal current passing through a coil can produce a dynamic magnetic field which also changes at the same frequency. When an electrode containing an inductor coil is present in this magnetic field, an induced current is generated in the inductor coil. Consequently, by detecting the induced current, the location of the electrode can be determined.

A pressure field based on an acoustic wave can also facilitate measurement of a sensing element's motion. An ultrasonic wave is directed to a tissue region. The ultrasonic wave can easily propagate through the tissue. A moving sensing element within the tissue may receive the ultrasonic wave with a Doppler frequency shift. As a result, by measuring the amount of Doppler frequency shift or time of arrival of the acoustic energy, the direction and velocity of the electrode's movement can be determined.

Continuous field tomography can be based upon measurement of the amplitude, frequency, and phase shift of the induced signal. When the external field is an electrical field or a magnetic field, the induced signal's amplitude is the main property for consideration in representative aspects. When the external field is a pressure field, the induced signal's frequency is the main property for consideration in representative aspects.

In further describing the subject invention, aspects of the data derived from a continuous tomography process, e.g., metrics such as cardiac-related parameters derived via an electrical tomographic method, are reviewed in greater detail first. Next, clinical data derived from, or otherwise associated with, the metrics, are described in greater detail. Next, illustrative examples of derivation of cardiac-related parameters and clinical data, and the utility thereof, are provided.

Various aspects of the present invention are associated with a tomographic method and/or system such as electrical tomography. The electrical tomography data obtained using electrical tomography methods and systems, e.g., as described above, may be employed raw or processed as desired, e.g., depending on the particular application for which the data are being employed. In certain aspects, electrodes, e.g., multi-electrode lead(s), can be placed in the heart. The electrodes may be connected to a receiver which can be employed to measure cardiac parameters of interest, e.g., blood temperature, heart rate, blood pressure, movement data, including synchrony data, as well as pharmaceutical therapy compliance. The obtained data may be stored in the receiver. Further, in certain aspects, the motion of one or more electrodes, e.g. one or more electrodes on the same cardiac lead or one or more electrodes on different cardiac leads, can be evaluated.

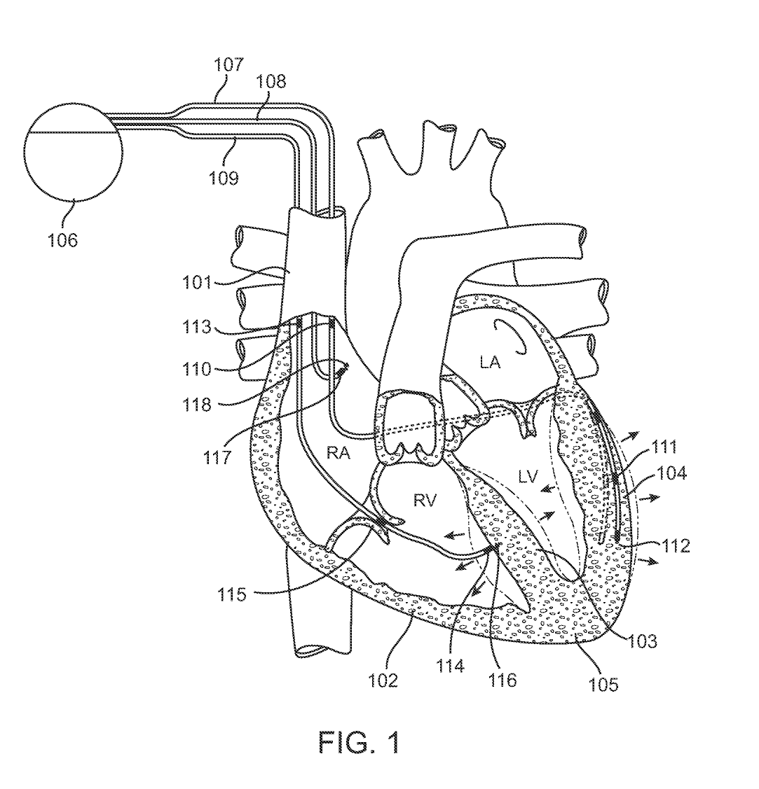

FIG. 1 provides a cross-sectional view of a heart with a cardiac timing device, according to an aspect of the present invention. The cardiac timing device may include, for example, a pacemaker 106, a right ventricle electrode lead 109, a right atrium electrode lead 108, and a left ventricle cardiac vein lead 107. Also shown are a right ventricle lateral wall 102, an interventricular septal wall 103, an apex of the heart 105, and a left ventricle cardiac vein 104. A skilled artisan will appreciate that various devices may be employed in addition to the pacemaker 106, e.g., an external device, an external circuit, an internal circuit, etc.

The left ventricle cardiac vein lead 107 is comprised of a lead body and one or more electrodes, e.g., a proximal electrode 110, a distal electrode 111, and a distal electrode 112. The distal electrodes 111 and 112 are located in the left ventricle cardiac vein and provide regional contractile information about this region of the heart. Having multiple distal electrodes allows a choice of optimal electrode location for CRT and/or other therapies. The proximal electrode 110 is located in a superior vena cava 101 in the base of the heart. This basal heart location is essentially unmoving and, therefore, can be used as one of the fixed reference points for the cardiac wall motion sensing system.

The left ventricle cardiac vein lead 107 may be constructed with the standard materials for a cardiac lead such as silicone or polyurethane for the lead body, and MP35N for the coiled or stranded conductors connected to the electrodes 110, 111, 112. The electrodes 110, 111, and 112 may be constructed from various materials, e.g., a Pt—Ir alloy as in 90% platinum and 10% iridium. Alternatively, these device components can be connected by various systems, e.g., multiplex systems such as those described in published United States Patent Application publication nos.: 20040254483 titled “Methods and systems for measuring cardiac parameters”; 20040220637 titled “Method and apparatus for enhancing cardiac pacing”; 20040215049 titled “Method and system for remote hemodynamic monitoring”; and 20040193021 titled “Method and system for monitoring and treating hemodynamic parameters, the disclosures of which are herein incorporated by reference, to the proximal end of left ventricle cardiac vein lead 107. The proximal end of the left ventricle cardiac vein lead 107 connects to the pacemaker 106.

The left ventricle cardiac vein lead 107 is placed in the heart using standard cardiac lead placement devices which include introducers, guide catheters, guidewires, and/or stylets. Briefly, an introducer is placed into the clavicle vein. A guide catheter is placed through the introducer and used to locate the coronary sinus in the right atrium. A guidewire is then used to locate a left ventricle cardiac vein. The left ventricle cardiac vein lead 107 is slid over the guidewire into the left ventricle cardiac vein 104 and tested until an optimal location for CRT is found.

The right ventricle electrode lead 109 is placed in the right ventricle of the heart and comprises an active fixation helix 116 at its end. The active fixation helix 116 is embedded into the cardiac septum. In FIG. 1, the right ventricle electrode lead 109 is provided with multiple electrodes 113, 114, and 115. The distal tip of the right ventricle electrode lead 109 is provided with the active fixation helix 116 which is screwed into the interventricular septal wall 103 or mid-septum.

The right ventricle electrode lead 109 is placed in the heart in a procedure similar to the typical placement procedures for cardiac right ventricle leads. The right ventricle electrode lead 109 is placed in the heart using the standard cardiac lead devices which include introducers, guide catheters, guidewires, and/or stylets. The right ventricle electrode lead 109 is inserted into the clavicle vein, through the superior vena cava 101, through the right atrium and down into the right ventricle. The right ventricle electrode lead 109 is positioned under fluoroscopy into the location the clinician has determined is clinically optimal and logistically practical for fixating the right ventricle electrode lead 109 and obtaining motion timing information for the cardiac feature area surrounding the attachment site. Under fluoroscopy, the active fixation helix 116 is advanced and screwed into the cardiac tissue to secure the right ventricle electrode lead 109 onto the septum.

Once the right ventricle electrode lead 109 is fixed on the septum, the right ventricle electrode lead 109 provides timing data for the regional motion and/or deformation of the septum. The electrode 115 which is located more proximally along the right ventricle electrode lead 109 provides timing data on the regional motions in those areas of the heart. By example, the electrode 115 situated near the atrioventricular (AV) valve, which spans the right atrium in the right ventricle, provides timing data regarding the closing and opening of the valve. The electrode 113 is located in the superior vena cava 101 in the base of the heart. This basal heart location is essentially unmoving and therefore can be used as one of the fixed reference points for the cardiac wall motion sensing system.

The right ventricle electrode lead 109 is typically fabricated as a soft flexible lead with the capacity to conform to the shape of the heart chamber. The only fixation point in this aspect of the present cardiac timing device is the active fixation helix 116 which is attaching the right ventricle electrode lead 109 to the cardiac septum. The right atrium electrode lead 108 comprising an electrode 117 is placed in the right atrium using an active fixation helix 118. The electrode 117, e.g., a distal tip, is used to provide both pacing and motion sensing of the right atrium. The above-described configuration is illustrative only. A skilled artisan will recognize that various electrode leads, electrodes, and/or placement configurations are possible.

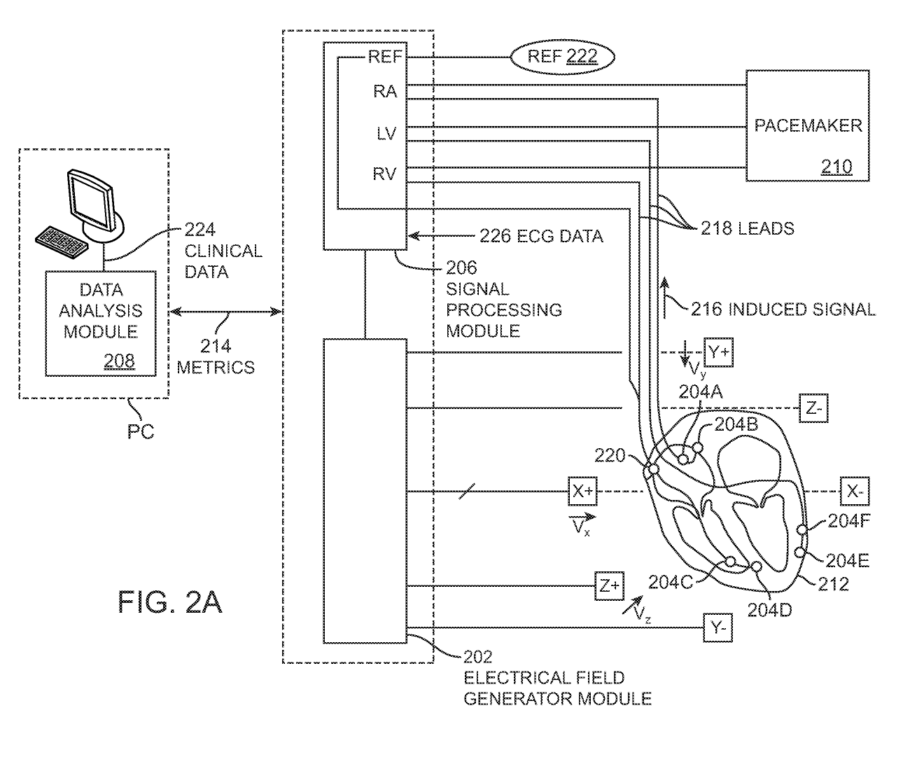

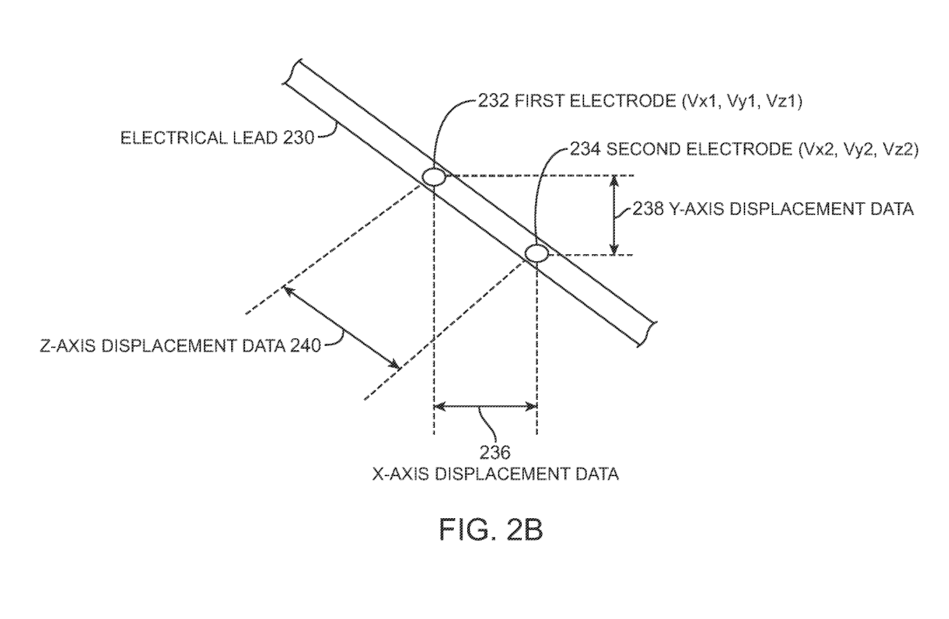

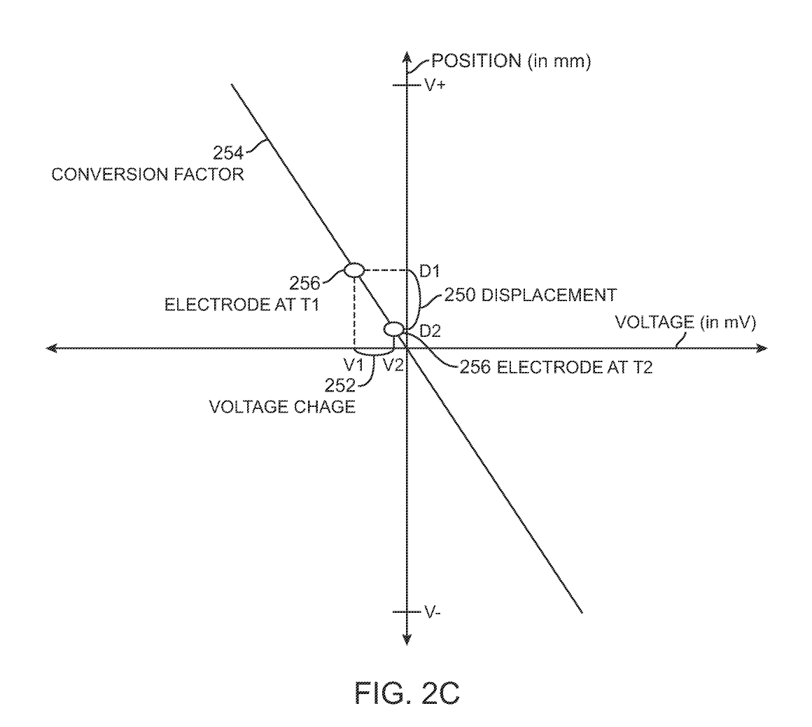

FIG. 2A illustrates an exemplary electrical tomography (ET) system, according to an aspect of the present invention. In FIG. 2A, the electrical tomography system comprises an electrical field generator module 202, one or more electrodes 204A-F, a signal processing module 206, and a data analysis module 208. The system may work in parallel with an existing pacemaker 210.

The electrical field generator module 202 generates one or more continuous electrical fields, e.g., in any orientation, and applies them to a subject (e.g., a patient) during an electrical tomography process. The electrodes 204A-F are stably positioned on several tissue sites within an internal organ, e.g., in the right atrium (RA), left ventricle (LV), and/or right ventricle (RV) of a heart 212, of the subject. In one aspect, the continuous electrical fields, e.g., vₓ, v_{y}, v_{z}, etc., comprise three orthogonal electrical fields along X-axis, Y-axis, and Z-axis.

In FIG. 2A, an AC voltage may be applied to generate vₓ through a pair of driving electrodes, e.g., X+, X−, which may reside external or internal to the subject's body, in the x direction. Similarly, v_{y} and v_{z} may be generated in the y direction through a pair of driving electrodes, e.g., Y+, Y−, and in the z direction through a pair of driving electrodes, e.g., Z+, Z−, respectively. Each of the vₓ, v_{y}, and v_{z} may operate at a different frequency. As a result, three induced signals, e.g., voltages, may be present on each of the electrodes 204A-F. Each induced signal also has a different frequency corresponding to the frequency of the electrical fields, e.g., vₓ, v_{y}, v_{z}, etc., in each direction. It is appreciated that the signals induced by the electrodes 204A-F may change as they travel via the electrical fields, e.g., from the high positive voltage close to positive driving electrode to the high negative voltage close to negative driving electrode. Therefore, by detecting the three induced signals using the signal processing module 206, the locations of the electrodes 204A-F can be determined in a three dimensional space.

In addition, the signal processing module 206 generates and forwards one or more metrics 214 associated with the electrodes 204A-F based on signals 216 induced and forwarded by the electrodes 204A-F in response to the continuous electrical fields. In one aspect, the metrics 214 may comprise displacement data of the electrodes 204A-F and/or their respective temporal data. As illustrated in FIG. 2A, leads 218 are used to forward signals from the electrodes 204A-F to the signal processing module 206. In one aspect, the induced signal 216 may be forwarded to the signal processing module 206 wirelessly if wireless transmitters associated with the electrodes 204A-F and/or the leads 218 are implemented in the ET system of FIG. 2A.

Multiplexing may be required if the signal processing module 206 e.g., embodied as a receiver, does not have enough channels or computing power to simultaneously process data from X, Y, and Z directions for more than one electrode. Time multiplexing requires the signal processing module 206 to switch between the electrodes 204A-F. For instance, the signal processing module 206 may look at a right-ventricular distal electrode for 1 msec and then switch to look at a left-ventricular distal electrode for 1 msec. Likewise, frequency multiplexing may result in looking at a signal axis of different frequencies at a time.

Additionally, an electrode 220 may be used as a reference port, which may couple to an external voltage reference point 222, such as ground. The data analysis module 208, which may be an application executable on a computer, e.g., a PC, a laptop, etc., then generates clinical data 224 based on the metrics 214.

As illustrated in FIG. 2A, the pacemaker 210 can send regular pacing signals to the electrodes 204A-F while the electrical tomography process is performed. Such simultaneous operation may be possible where short pulses are used as pacing signals, whereas constant sinusoidal signals with well defined frequencies are used as the driving voltages. In one aspect, the data analysis module 208 may generate configuration parameters for optimizing the operation of the pacemaker 210 based on the clinical data 224. Then, the pacemaker 210 may be reconfigured to optimize its operation. It is appreciated that the system described herein is operable without the pacemaker 210.

In one aspect, electrocardiogram (ECG) data 226 of the subject may be processed in the signal processing module in parallel with the metrics 214 to assist the analysis of the induced signals 216, e.g., to identify a start of cardiac contraction of the heart.

It is appreciated that the system illustrated in FIG. 2A can be used to perform a similar operation on other internal organs and/or systems, e.g., the internal organ can be one of adrenals, appendix, bladder, brain, eyes, gall bladder, intestines, kidney, liver, lungs, esophagus, ovaries, pancreas, parathyroids, pituitary, prostate, spleen, stomach, testicles, thymus, thyroid, uterus, veins, etc. It is also appreciated that the electrical field generator module 202, the signal processing module 206, and the data analysis module 208 can be integrated and/or implemented in a single device or as a combination of individual devices. For instance, the ET signal processing device and metric derivation performed by the signal processing module 206 can occur in the pacemaker 210, or the ET signal processing can occur on a chip (e.g., which contains the signal processing module 206) on any one of the leads 218.

FIG. 2B illustrates an exemplary process of calibrating a voltage to displacement conversion factor, according to one aspect of the present invention. The voltage to displacement conversion factor may be based on a ratio of a displacement to a voltage change, e.g., in a unit of mm/mV. Thus, a change in voltage due to a movement of an electrode in an electrical field of the ET system in FIG. 2A can be translated into displacement data using the conversion factor. In one exemplary implementation, inter-electrode spacing on an electrical lead can be used to obtain the conversion factor. This can be done by measuring the voltage difference between adjacent electrodes on the electrical lead. Then, with knowledge of the inter-electrode spacing or set distance between the two adjacent electrodes, the conversion factor can be computed.

As illustrated in FIG. 2B, in the case of an electrical lead 230 with more than two independent electrodes, e.g., tetra electrodes, the voltages induced by one or more electrical fields may be used to determine the conversion factor. For example, knowing locations of a first electrode 232 and a second electrode 234, X-axis displacement data 236, Y-axis displacement data 238, and/or Z-axis displacement data 240 may be obtained. Then, the conversion factors for electrical fields in x, y, and z directions may be obtained using respective displacement data and induced voltages measured at the first electrode 232 and the second electrode 234, respectively. That is, the voltage to displacement conversion factor for the electrical field in x-direction can be obtained by dividing the X-axis displacement data 236 with (Vx1-Vx2). Likewise, the voltage to displacement conversion factor for the electrical field in y-direction can be obtained by dividing the Y-axis displacement data 238 with (Vy1-Vy2), and the voltage to displacement conversion factor for the electrical field in z-direction can be obtained by dividing the Z-axis displacement data 240 with (Vz1-Vz2). Thus, the calibration process can be used to obtain more precise measurement of displaced electrodes using the calibrated voltage to displacement conversion factor obtained in the process illustrated in FIG. 2B.

FIG. 2C illustrates an exemplary method for measuring a displacement 250 of an electrode 256 due to a change in induced voltage (e.g., a voltage change 252) of the electrode 256, according to an aspect of the present invention. In FIG. 2C, the electrode 256 at T1 refers to a first moment when an induced voltage V1 of the electrode 256 due to the electrical field is measured at T1. Likewise, the electrode 256 at T2 refers to a second moment when an induced voltage V2 of the electrode 256 due to the electrical field is measured at T2. Additionally, using the slope of the graph as a conversion factor 254 and the two induced voltages V1 and V2, the displacement 250 (D2-D1) of the electrode 256 between the two different instances T1 and T2 can be obtained.

In order to calculate the displacement 250, the electrical tomography system needs to be configured. In one exemplary aspect, the electrical field generator module 202 may need to be field balanced, where the field balancing refers to the process of adjusting strengths of positive and negative drive electrodes in order to center the electrical field on the electrodes. It is appreciated that in a homogenous, ideal model of the subject's torso, the applied electrical field may vary linearly with distance from each drive electrode, crossing zero volt at half-way point between the positive and negative drive electrodes. However, in reality, the electrical field may be non-linear and/or distorted due to the non-homogenous nature of an organ, tissues, varying fluid volumes, etc. of the subject's body through which the electrical field travels during the electrical tomography process. Thus, the field balancing may be performed to adjust the drive strengths of the positive and negative electrodes so that the measured or induced voltages at the electrodes are close to zero, e.g., the electrode at T1 256. This may signify that the electrical field is centered about the heart.

In another exemplary aspect, amplitude balancing may be the process of increasing the drive strengths of positive and negative electrodes to augment gain in the induced voltage since, in an ideal electrical field generator, the gain may be determined by the overall drive strength, e.g., V+-V−, of the two drive electrodes as well as the distance between them. Accordingly, the overall drive strength and distance may be increased to obtain the optimal gain while avoiding saturation of the induced voltage. This may be achieved by increasing the drive strength in a step-wise fashion while ensuring that there is no saturation in the measured voltage.

In yet another exemplary aspect, phase balancing may refer to selection of a phase for the driving signal generated by the electrical field generator module 202 of FIG. 2A that results in the largest peak-to-peak amplitude signal received at the signal processing module 206, where the signal is an amplitude modulated (AM) sine wave signal. Since the frequency and phase of the signal need to be determined to demodulate the AM signal at the signal processing module 206, the phase can be selected by iterating through various phases and comparing the demodulated signals which correspond to changes in the phase. Then, the phase that results in a signal with the largest peak-to-peak amplitude may be selected. It is appreciated that the phase balancing may not be necessary if the electrical tomography system is based on a digital system or if quadrature demodulation is performed, e.g., AM demodulation at two phases separated by 90 degrees.

Furthermore, frequency sweeping may refer to the process of scanning frequency bands for noise-free or low in-band noise frequency bands. The process may be used to select a particular frequency, e.g., a frequency nearly free of interference. The interference, for example, may include noises from patient monitoring equipment, other medical devices, external sources, etc. This can be done, for example, by studying data from the electrodes without any electrical fields applied and then looking at the frequency spectrum for areas with relatively low spectral power. Once these regions are identified, the frequency of the drive electrodes is set to correspond to the lowest noise regions. Moreover, pace-pulse blanking may refer to the process of removing artifacts due to the delivered stimulation pulse from the pacemaker, where the pulse often distorts the drive signal by causing narrow but large amplitude spikes.

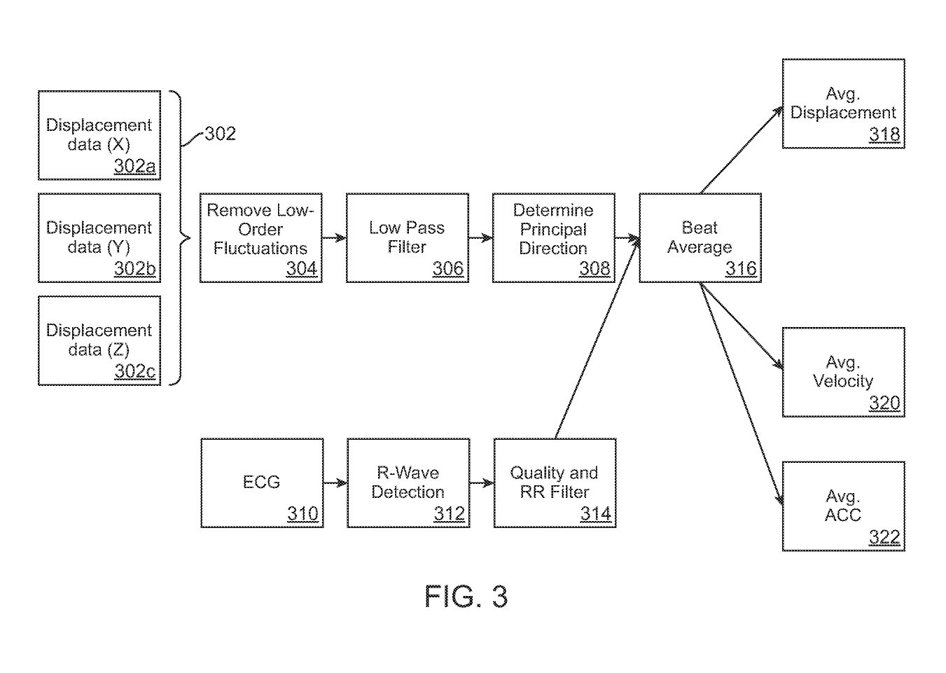

FIG. 3 illustrates an exemplary data analysis process, according to an aspect of the present invention. It is appreciated that the data analysis process may be implemented by an application, e.g., the data analysis module 208 of FIG. 2A. The metrics 214 from each electrode, e.g., 204A-F, in each of the x, y, and z axes, may be received via respective data channels at step 302. At step 304, the metrics, e.g., displacement data along X-axis 302a, displacement data along Y-axis 302b, and displacement data along Z-axis 302c, and their respective temporal data, may be processed to remove low-order fluctuations, such as respiratory effects. The respiratory effects include, for example, thoracic impedance that may change with respiratory cycle and cause large amplitude fluctuations in the acquired electrical tomography (ET) signal. General linear modeling techniques may be used to estimate the respiratory phase and remove low-order fluctuations in the signal at step 304. Additional techniques include median filtering, fitting of low-order polynomials, or high-pass filtering. Cardiac performance and motion can be modulated by respiration. Therefore, knowledge of respiratory phase would allow comparison of derived metrics across different respiratory states.

A low-pass filter may be applied to remove unwanted physiologic frequencies at step 306. From the combined x, y, and z axes data sets, a principal direction, e.g., a three-dimensional direction of maximal displacement of the electrodes, can be computed at step 308. Velocities and accelerations of the electrodes can be computed from the measured displacement along the principal direction, respectively.

Electrocardiogram (ECG) data can be used at step 310 in parallel with the metrics or displacement data to identify the start of each individual cardiac contraction. The R-wave detection may be used at step 312 to detect the peak of the QRS complex, which is the portion of the electrocardiogram comprising the Q, R, and S waves, together representing ventricular depolarization. Beats within a specified narrow intra-beat interval, which is denoted by the time between two consecutive R-waves or RR interval, may be used at step 314 to minimize the effect of modulation of the ET data related to fluctuation in the RR interval. Information about the temporal location of beats of interest may be used at step 316 to generate an average displacement at step 318, an average velocity at step 320, and/or an average acceleration ET trace at step 322.

In one aspect, the data may be employed, either alone or in combination with non-electrical tomography (ET) data, such as data obtained from other types of physiological sensors, e.g., pH sensors, pressure sensors, temperature sensors, etc., to determine one or more physiological parameters of interest, such as cardiac parameters of interest.

Parameters of cardiac performance measured using this approach can be measured both directly and indirectly. Examples of parameters which can be directly measured include, but are not limited to: cardiac wall motion, including measurements of both intra-ventricular and inter-ventricular synchrony; measurements of myocardial position, velocity, and acceleration in both systole and diastole; measurements of mitral annular position, velocity, and acceleration in both systole and diastole, including peak systolic mitral annular velocity; left ventricular end-diastolic volume and diameter; left ventricular end-systolic volume and diameter; ejection fraction; stroke volume; cardiac output; strain rate; inter-electrode distances; beat-to-beat variation; and QRS duration. Parameters which can be measured indirectly include, but are not limited to, dP/dt (a proxy for contractility); dP/dt(max); and calculated measurements of flow, which include mitral valve flow, mitral regurgitation, stroke volume, and cardiac output.

Other parameters which are helpful in management of cardiac patients include, but are not limited to, transthoracic impedance, cardiac capture threshold, phrenic nerve capture threshold, temperature, respiratory rate, activity level, hematocrit, heart sounds, and sleep apnea determination.

In one aspect, additional sensors, e.g. flow sensors, temperature sensors, pressure sensors, accelerometers, microphones, etc., may be used to obtain physiologic or cardiac parameters. Both the raw data obtained with this method and processed data can be displayed and used to evaluate cardiac performance, e.g., generate clinical data.

In one aspect, multiple parameters may be measured. Further, multiple clinical data may be derived from the parameters. Such parameters are discussed in detail in U.S. patent application Ser. No. 11/731,786 (published U.S. patent application no. 20080058656) titled “Electric Tomography” filed on May 30, 2007, which is hereby incorporated by reference in its entirety.

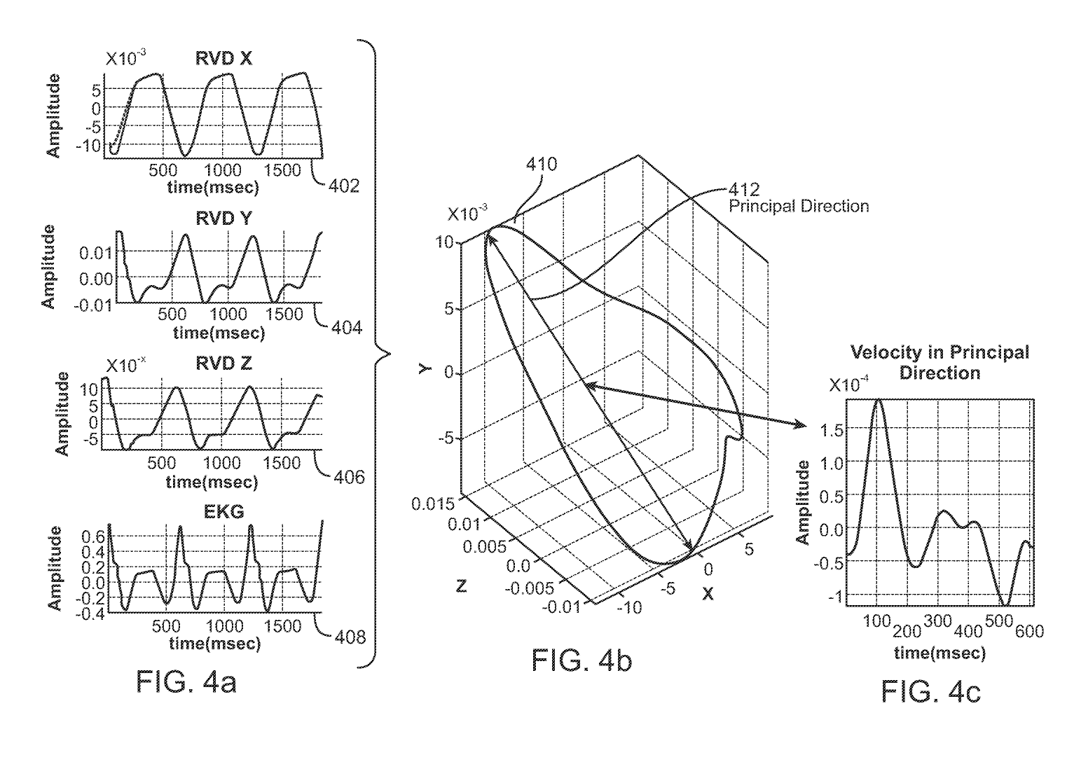

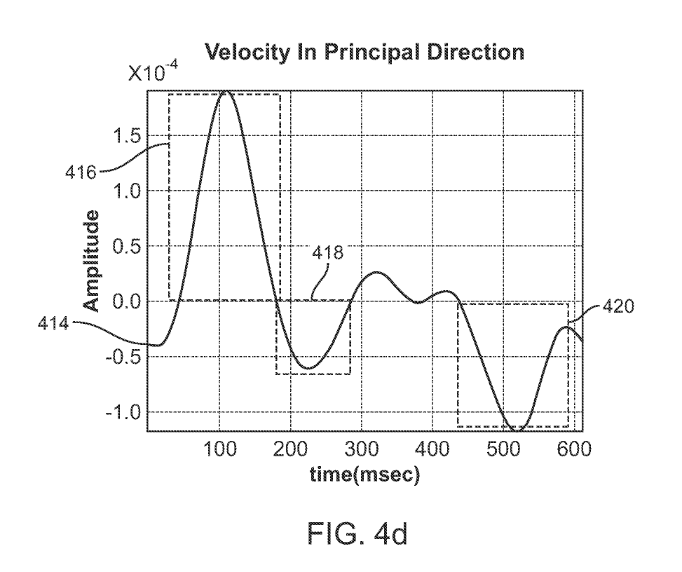

FIGS. 4a, 4b, and 4c illustrate two dimensional and three dimensional representations of clinical data, according to various aspects of the present invention. As shown in FIG. 4a, individual ET displacement data from each of the reference directions, e.g., right ventricular distal electrode (RVD) placed near the apical septum in each of the three reference directions RVD X, RVD Y, RVD Z, and an ECG or EKG, and time or temporal data can be used to generate a three-dimensional (3-D) representation of the electrode motion, e.g., 402, 404, 406, and 408.

In FIG. 4a, time averaging and/or beat averaging may be used to average together electrical tomography data from more than one cardiac cycle, e.g., three cardiac cycles. Although one can derive the ET metrics, e.g., S-velocity amplitude, for every single cardiac contraction, multiple cycles can be averaged together to create a higher signal-to-noise ratio (SNR), prior to reporting the ET metrics to the user, e.g., a clinician. In combination with the ECG, the start and end of each cardiac cycle may be identified by looking at the morphology of the ECG. Then, the heart motion may be measured with ET for ten to thirty seconds for a given pacing configuration, e.g., a combination of pacing location and timing. During this time, metrics can be generated on a beat-to-beat basis or the data can first be averaged over multiple beats to present to the physician. The clinician or automated systems can compare generated metrics for two or more pacing configurations and determine the best configuration. For instance, condition 1, e.g., RA, RV, or LV pacing with AV (atrioventricular) delay of 30 ms, has an S-Velocity amplitude of 8 m/sec while condition 2, e.g., RA, RV, or LV pacing with AV delay of 120 ms, has an S-Velocity amplitude of 12 m/sec. The clinician may choose, for example, condition 2 based on this metric related to the contractile performance of the heart.

The 3-d representation of the electrode motion, shown in FIG. 4b, is a function of the cardiac cycle and typically undergoes an elliptical path 410. The motion along the long axis of the ellipse is referred to as a principal direction 412 and corresponds to the direction of maximal displacement. Projection of the X, Y, and Z displacements along the vector of the principal direction and differentiation leads results in the derived principal velocity of the electrode, as illustrated in FIG. 4c. It is appreciated that each, or any combination, of linear or non-linear X, Y, and/or Z displacement data can be used to describe the electrode movement, e.g., the motion of the tissue site where the electrode is attached. Accordingly, the projection of X, Y, and Z displacements can be performed onto any plane or axis to investigate specific motion modes and paths.

FIG. 4d illustrates physiologically meaningful morphology of the principal velocity graph of FIG. 4c, according to an aspect of the present invention. The principal velocity graph comprises a systolic (S) velocity wave 416, an early (E) diastolic velocity wave 418, and an atrial (A) contraction velocity wave 420. The principal velocity graph starts at peak of the R-wave 414 of ECG or the beginning of left ventricle (LV) contractile period. The morphology of the principal velocity has physiologic interpretation with the initial positive peak of the systolic velocity wave 416, the negative peak of the early diastolic velocity wave 418, and the negative peak of the atrial contraction velocity wave 420. These three morphologic points of interest, e.g., 416, 418, and 420, may be analogous to the S, E, and A waves of a typical of tissue-Doppler imaging (TDI) of the mitral valve annulus.

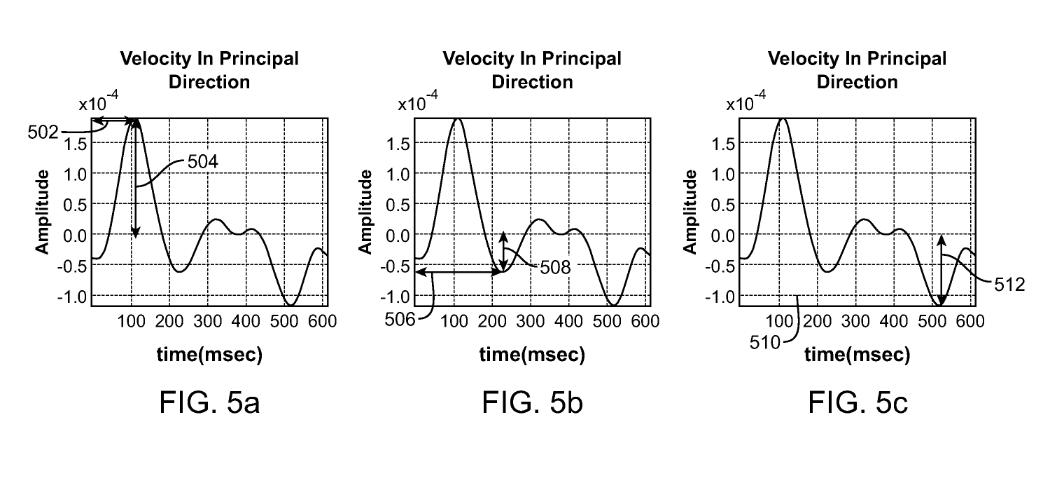

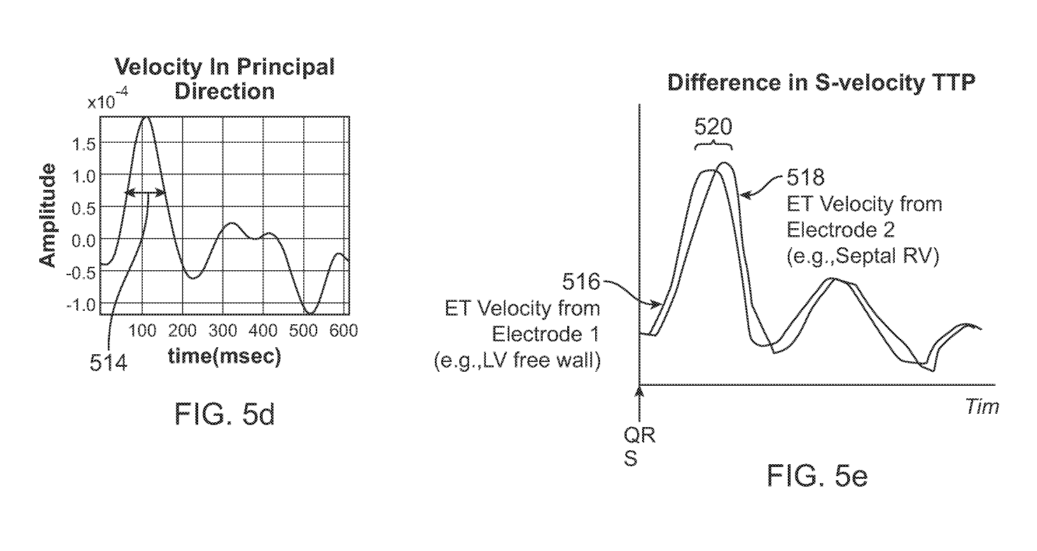

FIGS. 5a-5d illustrate interpretations of the ET velocity trace of FIG. 4d, according to various aspects of the present invention. In FIG. 5a, a peak amplitude 504 and a time-to-peak 502 of the systolic velocity wave 416 are shown. The peak amplitude 504 and time-to-peak 502 may be utilized as an indicator of systolic performance and contractile ability of the myocardium. An example of the clinical utility of this metric relates to the optimization of pacing therapy. In cardiac resynchronization therapy (CRT), nearly 30% of patients do not respond and many of the responders do not receive optimal therapy. ET metrics can be used to determine the optimal pacing configuration. For example, the peak amplitude 504 can be measured under various pacing configurations which can comprise a combination of RA, RV, and LV electrode position and relative timing, e.g., AV delay, VV (interventricular) delay.

Since the peak amplitude 504 and the time-to-peak 502 of the systolic velocity wave 416 are reflective of the underlying myocardial contractile performance of the LV, one can use this measure to determine optimal pacing therapy. With optimization of pacing therapy via ET, a greater proportion of patients may benefit from CRT. Further, the peak amplitude 504 and time-to-peak 502 may be utilized, for example, as a surrogate measure of LV dP/dT(max). Still further, the peak amplitude 504 and time-to-peak 502 may be utilized, for example, as a surrogate measure of TDI S-Velocity. LV dP/dt(max) and TDI S-Velocity have been previously shown to reflect myocardial contractile performance and can be utilized in a similar fashion as described above, to determine optimal pacing therapy.

In FIG. 5b, a peak amplitude 508 and a time-to-peak 506 of the early diastolic velocity wave 418 are shown. The peak amplitude 508 and time-to-peak 506 may be utilized, for example, as an indicator of passive filling of the LV and of diastolic performance and dysfunction. Diastolic performance is an important indicator of disease state. Worsening diastolic performance is often a leading indicator of negative hypertrophic cardiac remodeling. Increased LV stiffness and size resulting from diastolic dysfunction are hallmarks of progression of heart failure. Therefore, measurement of diastolic performance is important for understanding the progression of heart failure as well as for optimizing pacing and pharmaceutical therapy for optimal diastolic performance. Further, the peak amplitude 508 and the time-to-peak 506 may be utilized, for example, as a surrogate measure of LV dP/dT(min), TDI E-Velocity LV end-diastolic pressure-volume relationship, and LV stiffness.

In FIG. 5c, a peak amplitude 512 and a time-to-peak 510 of the atrial contraction velocity wave 420 are shown. The peak amplitude 512 and the time-to-peak 510 may be utilized as an indicator of atrial contractile performance and coordinated atrial/ventricular contraction. Decreased atrial contractile performance can be reflective of increased end-diastolic LV pressure and stiffness or disease progression of the left atrium or mitral valve, e.g., atrial fibrillation, mitral valve regurgitation, etc. Therefore, the peak amplitude 512 and the time-to-peak 510 may be useful for understanding disease progression and for optimizing pacing and pharmaceutical therapies for disease management. Further, the peak amplitude 512 and the time-to-peak 510 may be utilized, for example, as a surrogate measure of TDI A-Velocity, LA dP/dT(max), and LV pressure.

In FIG. 5d, a full-width half maximum 514 or aspect ratio of the systolic velocity wave 416 is shown. The full-width half maximum 514 may be utilized, for example, as an indicator of passive filling of the LV and diastolic performance and dysfunction. Further, the full-width half maximum 514 may be utilized, for example, as a surrogate measure of LV dP/dT(min), TDI E-Velocity, LV end-diastolic pressure-volume relationship, and LV stiffness. Metrics related to diastolic performance can be used as described above to determine disease state and drive optimal pacing and pharmaceutical therapy.

Direct observance of valvular events in the ET data may allow the clinician to more accurately determine relative timing in the cardiac cycle. For example, a surrogate measure for ejection period can be derived by looking at the breadth of the S-velocity peak, where the breadth can be obtained by measuring the time between zero crossings of the S-Velocity peak or the full-width half-maximum 514. It is appreciated that the ejection period is the time between aortic valve opening and aortic valve closure in which the blood in the left-ventricle (LV) is ejected. The shorter the ejection period, the more efficient and effective the contraction of the heart is. This metric would be used similarly to derive other metrics, and it may be minimized by adjusting the pacing configuration.

Additionally, isovolumic contraction interval may refer to the time in which the mitral valve closes and/or when the aortic valve opens. This may be the time where the cardiac muscles contract and begin to build up pressure in the left ventricular (LV). This can be derived by coupling information about the R-wave timing from ECG data of the heart with the start time of the S-velocity peak from the ET data.

FIG. 5e illustrates a difference in corresponding time-to-peaks (TTPs) 520 of two systolic (S) velocity waves for two electrodes located on two different tissue sites in the heart, according to an aspect of the present invention. In one aspect, a first electrode may be located on the LV free wall, whereas a second electrode may be located on the septal right ventricle (RV). Thus, the difference in time-to-peaks (TTP) 520 may be obtained by comparing the systolic velocity wave based on the first electrode 516 and the systolic velocity wave based on the second electrode 518. In one aspect, the difference 520 may be utilized, for example as an indicator of cardiac dysynchrony and coordinated contractile performance. Cardiac dysynchrony is often thought to be a driving mechanism for progression of heart failure. Furthermore, reducing dysynchrony with CRT has been shown to lead to positive remodeling of the LV in heart failure patients. Discoordinated contraction results in decreased contractile performance and a poorer quality of life for patients. By reducing dysynchrony through optimal pacing therapy as determined with the use of ET metrics of dysynchrony, contractile performance may be increased acutely in addition to possible positive remodeling of the LV.

The relative timing of morphological features of ET derived velocity from two or more locations can be used to quantify synchrony of the heart. If the S-velocity time-to-peaks are similar from various regions of the heart (e.g., LV leads, RV leads, etc.), this can signify a coordinated and synchronous cardiac contraction. Systolic and diastolic synchrony can be computed using the standard deviation of the time-to-peak derived from multiple electrodes for S and E velocities, respectively. In one application, a clinician may attempt to maximize the synchrony by choosing a pacing configuration that has the minimum time-to-peak standard deviation for the S-velocity.

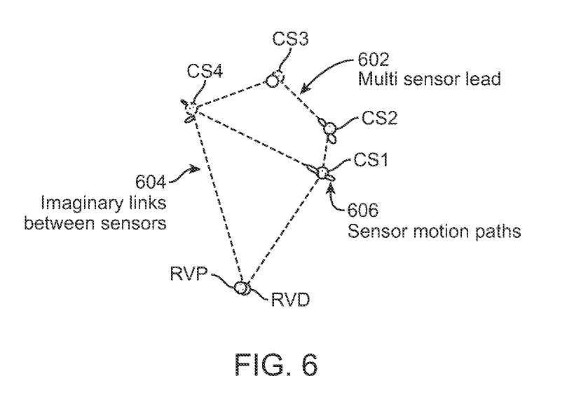

FIG. 6 illustrates a relative position of electrodes derived from electric tomography, according to an aspect of the present invention. In FIG. 6, a multi-sensor lead having electrodes (sensors) CS1-CS4, a right ventricle proximal electrode (RVP), and a right ventricle distal electrode (RVD) are shown, as well as imaginary links between sensors 604 and sensor motion paths 606. A relative position of the electrodes derived from ET allows computation of volume and dimension, e.g., an LV volume and dimension. As the RVP is in close proximity to the left ventricular apex, calculating the volume outlined by the coronary sinus electrodes at the base of the heart and the RVD at the apex can provide a measurement of LV volume. Similar methods employing the same or other electrodes can be used to measure the volumes of other cardiac chambers of interest. By detecting changes in volumes or distances defined by some or all of the electrodes, a variety of different cardiac function parameter may be determined.

The LV dimension may provide a useful measure of progression of heart failure. For example, the LV size reflects of negative hypertrophic remodeling and worsening of heart failure. Therefore, measurements of the LV dimension are important in diagnosing and monitoring the progression of heart failure in patients. For instance, the end-diastolic volume is reflective of the progression of heart-failure. The end-diastolic pressure-volume relationship is also used to describe the compliance of the LV, and its value is important for understanding diastolic heart-failure. This type of feedback may allow caregivers to understand the progression of heart disease and adjust its therapy accordingly. ET velocity can be projected into any arbitrary direction. Additionally, the ET derived volume measurements can be used analogously to other derived volume measurements, e.g., ultrasound, CT, MRI, conductance catheter, for assessment of LV performance.

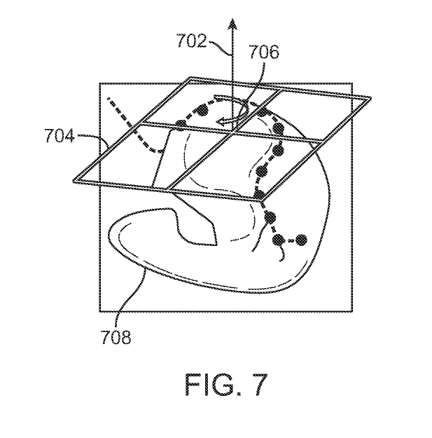

FIG. 7 illustrates a direction 702 defined normal to a mitral annular plane 704 toward an apex 706 of a heart 708, according to an aspect of the present invention. An ability to measure velocities in anatomically important directions may be important for characterization of systolic and diastolic performance.

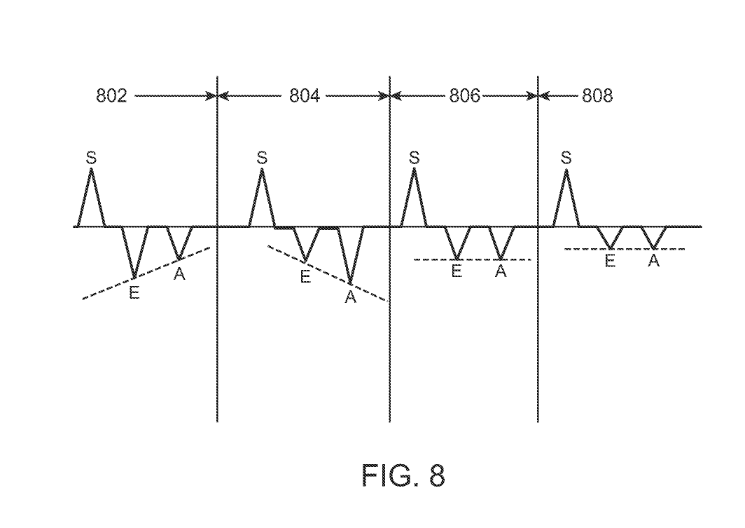

FIG. 8 illustrates interpretations of an exemplary ET velocity trace, according to an aspect of the present invention. The systolic velocity, or S-wave (S), the early diastole or E-wave (E), and the atrial contraction or A-wave (A), can be compared and clinical inferences can be drawn from the comparative relationships. The clinical inferences, for example, include an inference of diastolic dysfunction. The diastolic dysfunction is related to an inability of the LV to effectively fill with oxygenated blood from the left atrium (LA). The impaired ability may result in decreased cardiac output and negative hypertrophic remodeling of the left ventricle (LV). Increased LV stiffness is a hallmark of the worsened diastolic dysfunction.

For example, a configuration of E and A, as shown in 802, may indicate a normal diastolic function. A decrease in the E velocity and an increase in the A velocity, thus a change in the E/A ratio from the normal relationship of ET data, may be early indicators of various types of dysfunction, e.g., the change in the E/A ratio may indicate impaired relaxation as shown in 804. A decrease in the E velocity only as in 806 may indicate pseudo-normal functionality. Decreases both in the E velocity and the A velocity as shown in 808 may indicate restrictive functionality. The early indications of diastolic dysfunction, as provided by aspects of the present invention, may allow clinicians to adjust pacing or other therapies to optimally treat the dysfunction.

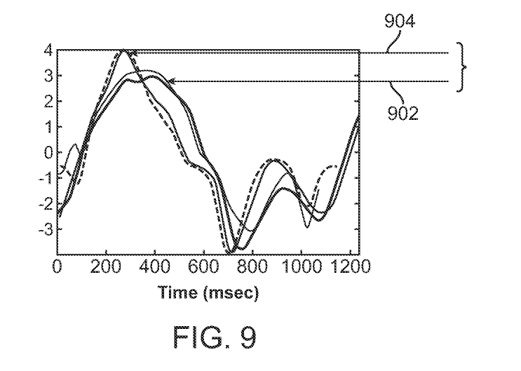

FIG. 9 illustrates an analysis of the impact of an LV pacing location, according to an aspect of the present invention. It is appreciated that data from human studies provided various data for analysis. Analytical conclusions regarding an impact of LV pacing location; a comparison to dP/dT(max); and a strong correlation between ET S-velocity amplitude and dP/dT (max) are included.

More particularly, in the human studies, the ET velocity was quantified from a standard RV electrode attached to the septal RV. Baseline curves 902, e.g., baseline 1 and baseline 2, reflect a characteristic motion without biventricular pacing whereas paced waveforms are from bi-ventricular pacing in either a bipolar, e.g., LV bipolar, or unipolar configuration for an LV pacing lead 904. Increased S-velocity and decrease in time-to-peak (TTP) with pacing reflects increased contractile performance with respect to the baseline curves 902. As a surrogate measure of dP/dT(max), S-velocity peak and TTP can be used to assess cardiac performance and drive optimization of pacing therapy.

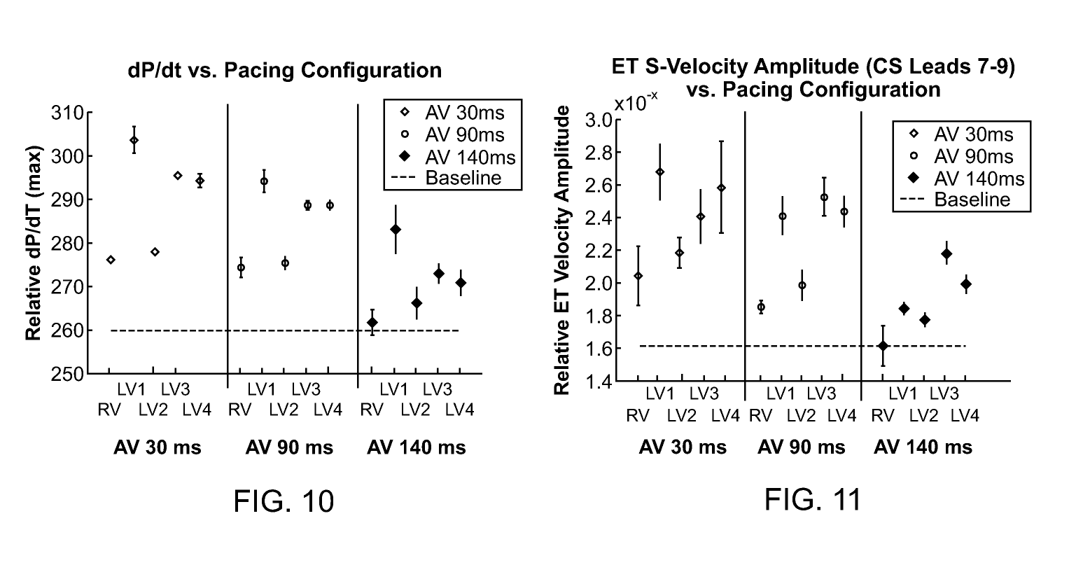

FIGS. 10 and 11 illustrate a dP/dT versus a pacing configuration and an ET S-Velocity amplitude versus a pacing configuration, according to an aspect of the present invention. As shown in FIGS. 10 and 11, dP/dT(max) changes with pacing configuration in the following manners:
(1) Lower AV delay results in higher dP/dT(max);
(2) At all AV settings, RV-only pacing has degraded performance;
(3) BiV pacing at distal LV electrode results in improved cardiac performance; and
(4) Nearly 20% difference between the max and min pacing configuration.

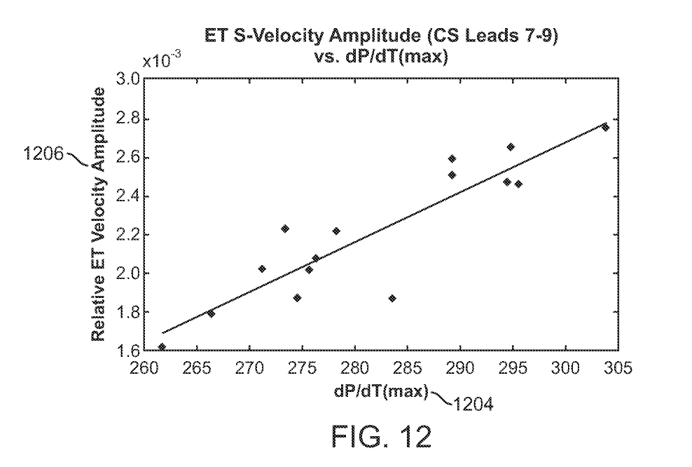

FIG. 12 illustrates a correlation between ET S-Velocity amplitude 1206 and dP/dT(max) data 1204, according to an aspect of the present invention. In one example, a result of a human clinical study, is shown in which the ET velocity metric S-velocity amplitude 1206 is derived for various biventricular pacing configurations. Pacing configuration comprises, for example, a programmed Atrial-Ventricular (AV) delay and an LV pacing site. The LV pacing site comprises pacing from four distal electrodes (spaced 7.5-15 mm. apart) of a temporary, customized Cardima catheter. Simultaneous LV pressure measurements are made with introduction of an LV pressure catheter. Modulation of dP/dT(max) 1204 is seen in FIG. 12 across pacing configurations, and the pattern is reflected in the ET S-velocity amplitude 1206. The resulting comparison yields a high correlation supporting the assertion that ET can accurately capture cardiac performance.

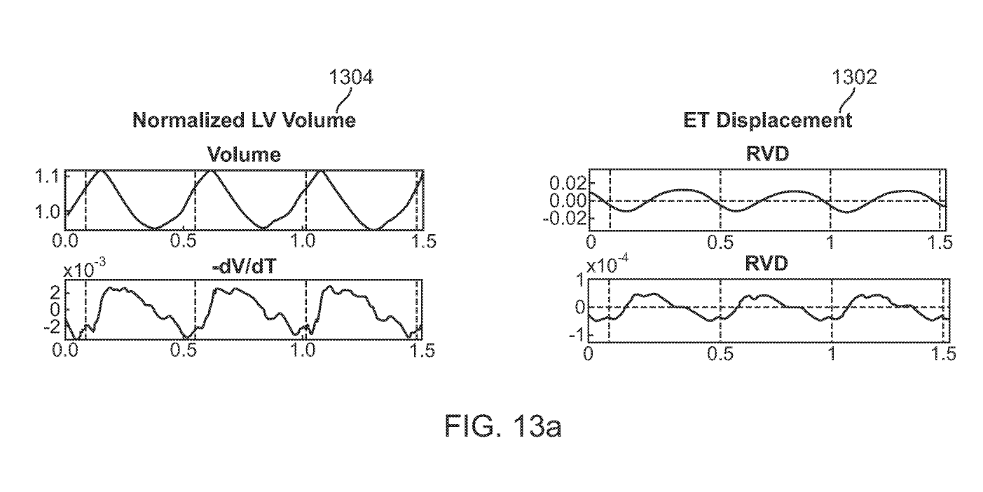

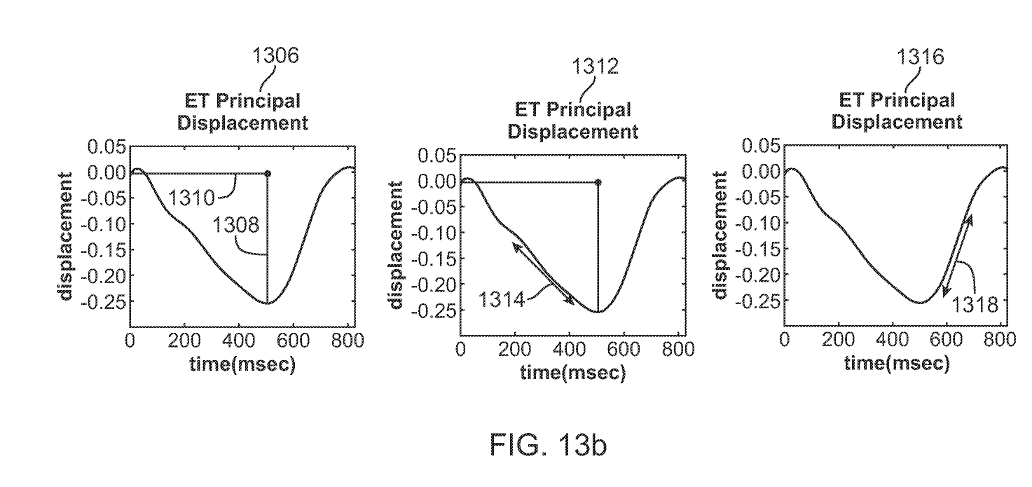

In one example, as illustrated in FIG. 13a, ET displacement data 1302 may be a surrogate measure for an LV volume metric 1304, according to an aspect of the present invention. The ET displacement data 1302 may be utilized, for example, to measure ejection fraction, LV stiffness, and LV chamber size. These measurements are useful in understanding disease progression and the impact of pacing and pharmaceutical therapies. In a feedback loop, they can be used to drive optimal treatment. Further, the effects of modulation with a pharmaceutical medication used to treat heart failure, e.g., dobutamine, a positive inotrope, etc., were measured.

In another example, as illustrated in FIG. 13b, peak displacement 1308 and time-to-peak 1310 of ET principal displacement 1306 may describe extent of myocardial wall contraction and provide a measure of systolic performance and contractile ability of myocardium. Accordingly, the metrics may be used as a surrogate measure of LV dP/dT (max), TDI S-Velocity, and/or cardiac wall strain measurements. In addition, slope 1314, e.g., time constant of systole, of the descending segment of ET principal displacement 1312 may be used to measure passive filling of the LV and diastolic performance and dysfunction. Thus, the metric may be used as a surrogate measure of LV dP/dT (max) and TDI S-Velocity. Furthermore, slope 1318 of the ascending segment of ET principal displacement 1316 may be used to measure passive filling of the LV and diastolic performance and dysfunction. Thus, the metric can be used as a surrogate measure of LV dP/dT (min), TDI E-Velocity, LV end-diastolic pressure-volume relationship, and LV stiffness.

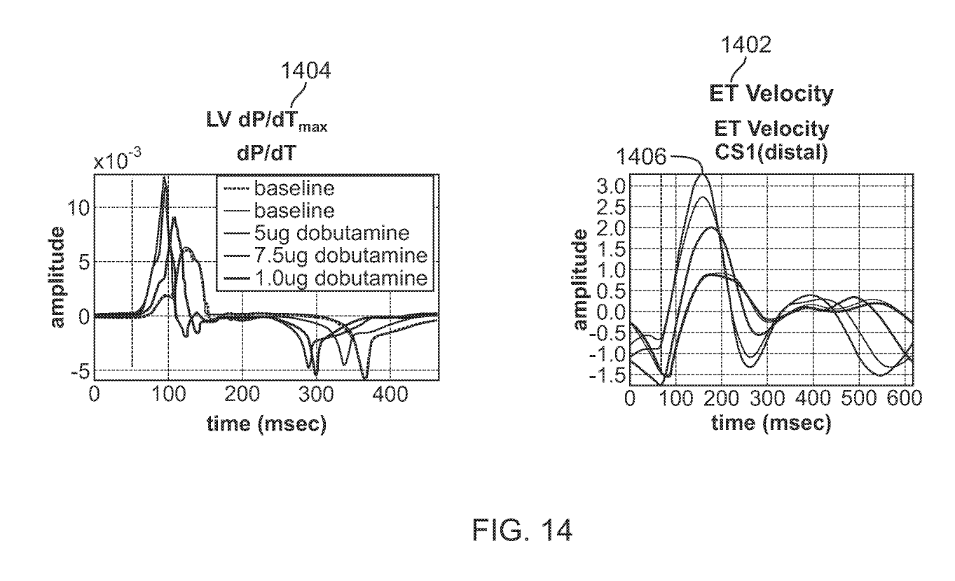

In one example, as illustrated in FIG. 14, ET S-Velocity data 1402 may be a surrogate measure for a dP/dT(max) metric 1404 in an animal model, according to one aspect of the present invention. More particularly, peak amplitude 1406 and time-to-peak (TTP) of the ET S-Velocity 1402 correlates well with dP/dT(max) in an animal model. Since dP/dT(max) is the gold standard measure of myocardial systolic performance, the peak amplitude 1406 and TTP may be utilized, for example, to assess underlying cardiac systolic performance.

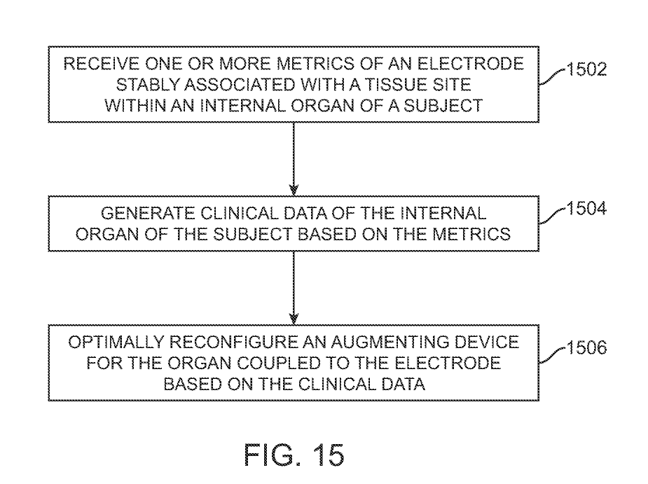

FIG. 15 is a process flow chart of an exemplary process for generating clinical data based on measurements or metrics obtained during an electrical tomography, according to an aspect of the present invention. In operation 1502, one or more metrics of an electrode stably associated with a tissue site within an internal organ of a subject are received, where the metrics are based on an induced signal of the electrode which is generated in response to one or more continuous electrical fields applied to the subject during an electrical tomography process. In operation 1504, clinical data of the internal organ of the subject is generated based on the metrics. In operation 1506, an augmenting device for the organ coupled to the electrode is optimally configured based on the clinical data. In one exemplary implementation, the augmenting device may be a pacemaker when the subjected internal organ is a heart.

One or more aspects of the subject invention may be in the form of computer readable media having programming stored thereon for implementing the subject methods or a computer system. The computer readable media may be, for example, in the form of a computer disk or CD, a floppy disc, a magnetic “hard card”, a server, or any other computer readable media capable of containing data or the like, stored electronically, magnetically, optically or by other means. Accordingly, stored programming embodying steps for carrying-out the subject methods may be transferred or communicated to a processor for execution, e.g., by using a computer network, server, or other interface connection, e.g., the Internet, or other relay means.

More specifically, computer readable medium may include stored programming embodying an algorithm for carrying out the subject methods. Accordingly, such a stored algorithm is configured to, or is otherwise capable of, practicing the subject methods, e.g., by operating an implantable medical device to perform the subject methods. The subject algorithm and associated processor may also be capable of implementing the appropriate adjustment(s). Of particular interest in certain aspects are systems loaded with such computer readable mediums such that the systems are configured to practice the subject methods.

The previous description of the disclosed aspects is provided to enable any person skilled in the art to make or use the present invention. Various modifications to these aspects will be readily apparent to those skilled in the art, and the generic principles defined herein may be applied to other aspects without departing from the spirit or scope of the invention. Thus, the present disclosure is not intended to be limited to the aspects shown herein but is to be accorded the widest scope consistent with the principles and features disclosed herein.

Although the invention is to some extent exemplified in terms of cardiac motion evaluation aspects, the invention is not so limited. The invention is readily adaptable to evaluation of movement of a wide variety of different tissue locations. The tissue location(s) are generally a defined location or portion of a body, e.g., subject, where in many aspects it is a defined location or portion, i.e., domain or region, of a body structure, such as an organ, where in representative aspects the body structure is an internal body structure and/or tissue, such as an internal organ, e.g., adrenals, appendix, heart, bladder, brain, eyes, gall bladder, intestines, kidney, liver, lungs, esophagus, ovaries, pancreas, parathyroids, pituitary, prostate, spleen, stomach, testicles, thymus, thyroid, uterus, and veins, etc.

What is claimed is:

1. A method for generating clinical data by processing at least one metric obtained via electrical tomography, the method comprising:
receiving at least one metric of a first electrode stably associated with a tissue site of a subject, wherein the at least one metric is based on an induced signal of the electrode generated in response to at least one continuous electrical field applied to the subject during an electrical tomography process; and
generating clinical data based on the at least one metric.

2. The method of claim 1, wherein the at least one continuous electrical field comprises three orthogonal electrical fields along an X-axis, a Y-axis, and a Z-axis.

3. The method of claim 2, wherein the at least one metric is based on displacement data along the X-axis, the Y-axis, and the Z-axis which correspond to the induced signal of the electrode, and the at least one metric is further based on temporal data associated with the displacement data.

4. The method of claim 3, wherein the generating the clinical data comprises determining a principal direction which is a maximal distance between any two points of the displacement data along the X-axis, the Y-axis, and the Z-axis.

5. The method of claim 4, wherein the tissue site is a cardiac tissue site.

6. The method of claim 5, wherein the generating the clinical data further comprises generating data representative of a principal velocity graph associated with the principal direction based on the displacement data and the temporal data, with the principal velocity graph comprising a systolic velocity wave, an early diastolic velocity wave, and an atrial contraction velocity wave.

7. The method of claim 6, further comprising analyzing a peak amplitude and a time to peak of the systolic velocity wave to determine a systolic performance or a contractile ability of a myocardium of the heart.

8. The method of claim 6, further comprising measuring a width between zero crossings of a peak or a full-width half maximum of the systolic velocity wave to determine a breadth of the peak of the systolic velocity wave.

9. The method of claim 6, further comprising analyzing a peak amplitude and a time to peak of the early diastolic velocity wave to determine at least one of a passive filling, a diastolic performance and a dysfunction of a left ventricle.

10. The method of claim 6, further comprising analyzing a peak amplitude and a time to peak of the atrial contraction velocity wave to determine an atrial contractile performance or a coordinated atrial/ventricular contraction.

11. The method of claim 6, further comprising analyzing a full-width half maximum of the systolic velocity wave to determine a passive filling, a diastolic performance, or a dysfunction of a left ventricle of the heart.

12. The method of claim 6, further comprising comparing the systolic velocity wave associated with the electrode and a second systolic velocity wave of a second electrode used for the electrical tomography to determine a cardiac dysynchrony or a coordinated contractile performance of the heart.

13. A system for generating clinical data by processing at least one metric obtained via electrical tomography, the system comprising:
an electrical field generator module for generating at least one continuous electrical field and applying the electrical field to a subject during an electrical tomography process, wherein a first electrode is stably associated with a tissue site of the subject;
a signal processing module for generating and forwarding the at least one metric associated with the electrode based on an induced signal of the electrode in response to the at least one continuous electrical field; and
a data analysis module for generating clinical data based on the at least one metric.

14. The system of claim 13, further comprising a device communicatively coupled to the electrode, wherein the device is operable for reconfiguration based on the clinical data.

15. The system of claim 13, wherein the signal processing module comprises a demodulator for demodulating the induced signal, and wherein the induced signal comprises an amplitude modulated sine wave.

16. The system of claim 13, wherein the at least one metric is based on displacement data of the first electrode.

17. The system of claim 16, wherein the displacement data are obtained by converting the induced signal to a physical unit of distance using a conversion factor.

18. The system of claim 17, wherein the first electrode is one of a plurality of electrodes implemented on an electrical lead, and wherein the conversion factor is obtained using an inter-electrode spacing of the plurality of electrodes.

19. The system of claim 18, wherein the signal processing module comprises a multiplexer configured for performing at least one of time multiplexing and frequency multiplexing respective induced signals forwarded from the plurality of electrodes.

20. A computer readable medium having instructions that when executed by a processor of an electrical tomography system causes the electrical tomography system to perform a method of generating clinical data by processing at least one metric obtained via electrical tomography, the method comprising:
receiving at least one metric of a first electrode stably associated with a tissue site of a subject, wherein the at least one metric is based on induced signal of the electrode generated in response to at least one continuous electrical field applied to the subject during an electrical tomography process; and
generating clinical data of based on the at least one metric.
